(19) United States
(12) Patent Application Publication
(10) Pub. No.: US 2018/0085520 A1
(43) Pub. Date: Mar. 29, 2018

(54) WITHIN-TIME INFUSION MODES FOR INFUSION PUMPS

(71) Applicant: Smiths Medical ASD, Inc., Plymouth, MN (US)

(72) Inventors: Rick LEDFORD, Brooklyn Park, MN (US); Eric WILKOWSKE, North Oaks, MN (US)

(73) Assignee: Smiths Medical ASD, Inc., Plymouth, MN (US)

(21) Appl. No.: 15/563,372

(22) PCT Filed: Mar. 14, 2016
(86) PCT No.: PCT/US2016/022332

§ 371 Date: Sep. 29, 2017

Related U.S. Application Data
(60) Provisional application No. 62/140,168, filed on Mar. 30, 2015.

(51) Int. Cl. A61M 5/168; A61M 5/142

(52) U.S. Cl. CPC A61M 5/16804; A61M 5/16831; A61M 5/14228; A61M 2005/1403; A61M 2205/50; A61M 2205/3379; A61M 2205/502

(57) ABSTRACT

An infusion mode for an infusion pump is configured for the scheduling of infusions without requiring the clinician to hand-calculate and manually update the infusion pump, should a stoppage occur. Consideration of a flush time (the duration of time for the flush) and flush rate (the rate for the flush), as part of the infusion is further included in the infusion scheduling.

### TECHNICAL FIELD

Subject matter hereof relates generally to infusion pumps, and more particularly, to the scheduling of infusions on, for, or with infusion pumps.

### BACKGROUND

Infusion pumps are useful medical devices for managing the delivery and dispensation of therapeutic medications. Infusion pumps provide significant advantages over manual administration by accurately delivering medications over an extended period of time. Infusion pumps are particularly useful for treating diseases and disorders that require regular pharmacological intervention, including cancer, diabetes, and vascular, neurological, and metabolic disorders. They also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large volume, patient-controlled analgesia (PCA), elastomeric, syringe, enteral, and insulin pumps. Infusion pumps can be used to administer medication through various delivery methods, including intravenously, intraperitoneally, intra-arterially, intradermally, subcutaneously, in close proximity to nerves, and into an intraoperative site, epidural space or subarachnoid space.

Typically, infusion pumps are locally controlled via the programming of the individual infusion pump. For example, a physician can configure an infusion pump to execute a delivery profile that corresponds to a patient's treatment needs, or a patient can configure an infusion pump according to their individual requirements within pre-defined limits without the involvement of a physician. Generally, an infusion pump is programmed or configured according to certain physiological, pharmacokinetic, and operational parameters or limits that are often predefined.

As described in International Publication No. WO2014/210465, which is incorporated herein by reference in its entirety, a patient can have multiple, mutually exclusive, and serially scheduled activities, wherein one of the activities is an administration of a medication via an infusion from an infusion pump. It is common for an infusion to be required before a subsequent scheduled activity can occur. For example, a patient might often require an infusion before the patient receives an x-ray. As a result, timely completion of the infusion according to a defined schedule is commonly required.

When an infusion pump is programmed or configured, it can often become stopped or interrupted, intentionally or unintentionally, during operation. A stoppage can occur for any number of reasons, such as a pump occlusion, pump or infusion line failure, or other device issue. In other examples, the pump can be manually stopped due to a patient issue or other external concern. Typically, after an infusion has been so interrupted and stopped, the infusion is restarted and rescheduled based on a hand-calculated time and rate whereby a determination of the new infusion timing duration or “window” is made and a rate is determined according to that new infusion timing window. The timing duration or “window” can be affected by the scheduled activities for the patient. Because of the delay, such an interrupted infusion is often restarted and completed at a maximum rate allowed for the particular drug or infusate in order to fit the infusion within the schedule.

Infusing at the maximum allowable rate, which might technically be safe, can in some instances adversely affect the patient such as by causing irritation at the infusion site. Patients receiving maximum rate infusions can also react adversely to the amount of medication received due to the maximum rate, which may be much greater than an optimal rate. Further, because of such interruptive stopping and restarting, other infusions or medical procedures often need to be delayed or rescheduled, resulting in potentially myriad subsequent problems in scheduling for both the patient and healthcare provider. Moreover, the hand-calculation of the restarted infusion can often lead to infusion errors. Therefore, there is a need for an infusion pump, system, and associated methods that allow for the scheduling of infusions without requiring the clinician to hand-calculate and manually update the infusion process upon the occurrence of an intentional or unintentional interruption or stoppage of the pump.

### SUMMARY

Embodiments described or otherwise contemplated herein substantially meet the aforementioned needs. For example, a “within-time” infusion mode (the term “within-time” will be further described by example through this document) allows a healthcare provider (typically a busy nurse) to better manage his or her day by allowing for a specified planned duration for an infusion or series of infusions. In another embodiment, within-time can include the feedback time or time for therapeutic effect. For example, in a clinical case, a drug can be administered to a patient by a nurse. Then, the nurse waits for feedback or therapeutic effect to see what effect, if any, the drug has had on the patient. In practice, the nurse can evaluate vital sign changes or draw blood in order to evaluate the drug's therapeutic effect. Subsequently, based on the evaluation, the nurse can administer more of the drug, wait, or change the drug. Therefore, within-time can include not just the time for one infusion or one drug administration, but can include a treatment plan or portions of a treatment plan.

Similarly, clinical workflows or other clinical interactions can be considered in within-time. For example, a nurse or other medical professional can be limited by the time their work shift ends. Thus, an infusion can be administered within the time until the end of the nurse's shift. Similarly, within-time can include the time it might take a nurse to interface with another patient, before re-attending to the first patient. In another example, as described above, the time to get feedback for a drug that was previously administered can be included within-time. In another example, in an emergency situation, the transit time for a patient in an ambulance or helicopter can be included within-time. For example, an infusion might need to be completed during the transit time. In another example, the time for a trip to another medical procedure, such as an x-ray, MRI, or CT scan, can be included within-time. In another example, the time to complete physical therapy can be included within-time. In another example, in a clinical setting, the time prior to another medical procedure starting can be included within-time. For example, particular infusions often need to be completed prior to surgery. In still another example, the time to complete multiple drug therapies can be included within-time.

Therefore, “within-time” can comprise the time for any one infusion, one infusion plus a clinical step, one infusion plus a series of clinical steps, a series of infusions, a series of infusions plus a clinical step, or a series of infusions plus a series of clinical steps.

Embodiments of infusion modes allow for the scheduling of infusions without requiring the clinician to hand-calculate and manually update the infusion pump, should an interruption or stoppage occur. According to embodiments, consideration of a flush time (a duration of time for a flush of the infusion line) and flush rate (a rate for the flush), as part of the infusion is further included in the infusion scheduling. In an embodiment, a within-time infusion mode can automatically prompt for a flush syringe as part of an infusion workflow and automatically deliver within the constraints of the within-time mode.

For example, in a within-time infusion mode, a target delivery time and a rate limit can be specified, along with the desired infusion(s). Should the infusion pump be stopped for any reason during operation, the rate is automatically and dynamically varied upon restarting to meet the scheduled delivery time within the rate limit for the desired infusion(s). For example, the rate may need to be accelerated if the original rate does not meet the target delivery time upon restart. In other examples, the rate can remain the same if the original rate does still meet the target delivery time upon restart. In embodiments, a flush can also be included or considered in the scheduled delivery time. Therefore, within-time infusion modes allow the healthcare provider to generally deliver the infusion at a rate less than the allowed maximum for the drug, which minimizes the dangers or adverse effects to the patient and to the patient infusion site. Further, such scheduling helps to manage the healthcare provider's busy day.

For example, an infusion order may be for two intermittent infusions, with a recommend minimum infusion window of five minutes. There are two medications that are to be given sequentially on the same pump or different pumps. A nurse has scheduled both to be administered within an infusion window of one hour (which is in accordance with the recommended minimum window of five minutes). The first infusion is started and completes within 25 minutes, as constrained by a maximum allowable rate for the first medication. The nurse then has ten minutes to start the second infusion that will also run for a target of 25 minutes. According to embodiments, as will be further described, the administration of both infusions have therefore been completed within the target time, schedule wise, even if the infusion is stopped for brief periods, for whatever reason. In embodiments, the second infusion can automatically be started after confirmation of completion of the first infusion.

In another example, a particular delivery profile, delivery pattern, or delivery segment can be delivered within a particular time. Referring to Application No. PCT/US15/18462, which is incorporated herein by reference in its entirety, predefined delivery profiles, delivery patterns, or delivery segments can be delivered through an infusion pump. For example, a profile segment can be defined by a set of delivery shape parameters, and/or user-defined parameters, which can be used as an underlying template for execution of delivery profiles or segments. In an embodiment, delivery profiles or segments of profiles that accurately model natural, physical variances, such as profiles created using fourth degree polynomials, (i.e., f(x)=ax4+bx3+cx2+dx+e) can be utilized. The delivery profile, delivery patterns, or delivery segments can be modified by changing of the coefficients of the underlying polynomial equation, or by adjusting the curve of the profile, pattern, or segment. In embodiments, determinations of time and/or rate can be made in relation to these delivery profiles, delivery patterns, or delivery segments such that a particular profile, pattern, or segment is delivered within a time.

In an embodiment, an infusion pump comprises a control module configured to receive within-time infusion mode pump parameters, wherein one of the within-time infusion mode pump parameters is a completion time for an infusion; a pump control subsystem comprising a mode control engine configured to implement the within-time infusion mode pump parameters, including calculating an infusion restart rate based on at least the completion time for the infusion if the infusion pump becomes stopped; and a pumping mechanism operably coupled to a reservoir and configured to be controlled by the pump control subsystem.

In an embodiment, a method for controlling an infusion in an infusion pump comprises implementing a within-time infusion mode on an infusion pump via a mode control engine; receiving within-time infusion mode pump data, wherein the within-time infusion mode pump data includes a completion time; initiating an infusion on the infusion pump according to infusion parameters derived from the received within-time infusion mode pump data; detecting a stoppage of the infusion pump; calculating an infusion restart rate based on at least the completion time for the infusion; restarting the infusion on the infusion pump according to the calculated infusion restart rate; and completing the restarted infusion within the completion time.

In an embodiment, a pump control system comprises programmable circuitry configured to implement a within-time infusion mode on an infusion pump via a mode control engine; receive within-time infusion mode pump data, wherein the within-time infusion mode pump data includes a completion time; initiate an infusion on the infusion pump according to infusion parameters derived from the received within-time infusion mode pump data; detect a stoppage of the infusion pump; calculate an infusion restart rate based on at least the completion time for the infusion; restart the infusion on the infusion pump according to the calculated infusion restart rate; and complete the restarted infusion within the completion time.

In embodiments, within-time calculations can be included in systems utilizing a pinch-clamp and a rate adjuster to control an infusion rate with respect to time in a simple mechanical setup. For example, in such embodiments, a personal computer, desktop computer, tablet computer, portable phone, wristwatch, or other embodiment can control the rate adjuster for any one infusion, one infusion plus a clinical step, one infusion plus a series of clinical steps, a series of infusions, a series of infusions plus a clinical step, or a series of infusions plus a series of clinical steps.

In embodiments as used herein, a continuous delivery mode comprises an infusion that is programmed to deliver a dose per unit time.

In embodiments as used herein, an intermittent delivery mode comprises infusion that is programmed to deliver a specific dosage (volume of drug) over a specified time.

In embodiments, a delivery mode is a general term used to describe the primary mode of delivery of a maintenance delivery segment of an infusion. In embodiments, infusion pumps can comprise three primary delivery modes: continuous, intermittent, and variable rate.

In an embodiment, a delivery plan comprises an ordered collection of delivery segments, which together describe the intended pattern of drug delivery during an infusion in terms of volumetric rate as a function of time.

In an embodiment, a delivery segment is a generic name for any portion of an infusion which can include a maintenance or main segment of the delivery, and other delivery features such as loading dose, bolus, flush, etc. In embodiments, the maintenance segment is one of continuous, intermittent, or variable rate. In embodiments, Patient Controlled Analgesia (PCA), variable rate including a taper, step, and target controlled infusions (TCI) can be added.

In an embodiment, a delivery method is a category of fluid delivery during an infusion which has a specific clinical purpose with respect to the delivery method. Examples of delivery methods can include loading dose, clinician bolus, flush, and keep vein open (KVO).

In a feature and advantage of embodiments, a pump infusion mode is configured to allow a user to program an intermittent, virtual, continuous, or other infusion by entering the maximum time within which the delivery should be completed. In embodiments, such a maximum time can be called a within-time intermittent infusion. An intermittent infusion has a relatively high infusion rate that alternates with a low programmable infusion rate to keep the cannula and/or patient's vein open.

In another feature and advantage of embodiments, when a within-time intermittent infusion is programmed, the pump or one or more associated modules is configured to calculate the delivery rate based on the dose to be delivered and the amount of time during which the delivery should occur.

In another feature and advantage of embodiments, when a within-time intermittent infusion is programmed, the pump or one or more associated modules is configured to accept user input of a maximum allowable delivery rate.

In another feature and advantage of embodiments, when a within-time intermittent infusion is programmed and delivery is interrupted, the pump or one or more associated modules is configured to recalculate the delivery rate when delivery is restarted in order to complete the delivery, deliveries, therapy, or treatment within the time specified, without exceeding the maximum allowable rate.

In another feature and advantage of embodiments, when a within-time intermittent infusion is programmed and delivery is interrupted and the recalculated rate is higher than the maximum rate, the pump or one or more associated modules is configured to use the maximum delivery rate when delivery is restarted and allow the delivery time to extend beyond the set time. In embodiments, a user can be alerted to any changes in rate or recommendations to changes in rate. The user can then accept or stop the delivery at the recalculated rate. For example, a user can be alerted that the previously programmed schedule cannot be met, and that delivery can be restarted at a maximum rate. The user can thus be prompted to confirm that delivery at the maximum rate and/or beyond the previously scheduled time is acceptable. In embodiments, the user can simply stop the infusion if the time extension or recalculate rate is unacceptable.

In another feature and advantage of embodiments, when a flush is programmed for a within-time intermittent infusion, the pump or one or more associated modules is configured to include the flush volume and associated delivery time with the dosage when calculating the delivery rate.

In another feature and advantage of embodiments, when a flush is programmed for a within-time intermittent infusion, the pump or one or more associated modules is configured to reduce the time used for calculating the delivery rate by a preset time associated with changing the syringe. In other embodiments, the total time used for calculating the delivery rate can be reduced by a varying amount of time, depending on the type of syringe. In other embodiments, the total time used for calculating the delivery rate can be reduced by a time programmed by the user.

In another feature and advantage of embodiments, when a clinician pushes the flush manually, the pump or one or more associated modules is configured to allow the clinician to program the flush amount to add to the delivery total.

In another feature and advantage of embodiments, clinicians can be advised of conflicts between rate and volume limits with respect to increased medication concentration. For example, a drug can be commanded to be given at a first concentration within a certain time, without exceeding a total volume limit. According to embodiments, it can be determined that delivering the drug at the first concentration will exceed the total volume limit. A prompt can be provided with an option to increase the drug from the first concentration to a second concentration. In embodiments, the clinician can select yes to increase to the second concentration or run with the first concentration

In another feature and advantage of embodiments, a pump infusion mode is configured to allow a user to program a continuous infusion by entering the planned time within which the delivery should be completed. In embodiments, such a planned delivery time can be called a within-time continuous infusion. A continuous infusion generally comprises small pulses of infusion with the rate of these pulses depending on the programmed infusion rate. In embodiments, the rate, length, or duration of the pulses can depend on the type of infusion pump utilized.

In another feature and advantage of embodiments, when a within-time continuous infusion is programmed, the pump or one or more associated modules is configured to calculate the dose to be delivered based on the rate and the amount of time during which the delivery should occur.

In another feature and advantage of embodiments, when a within-time continuous infusion is programmed, the pump or one or more associated modules is configured to allow the user to enter a maximum allowable delivery rate. In other embodiments, a maximum allowable delivery rate can be preprogrammed into the pump or associated module. In still other embodiments, a maximum allowable delivery rate can be derived from a database or drug library.

In another feature and advantage of embodiments, when a within-time continuous infusion is programmed and delivery is interrupted, the pump or one or more associated modules is configured to recalculate the delivery rate when delivery is restarted. In such embodiments, the infusion is thereby completed with the calculated dose to be delivered within the time specified and without exceeding the maximum allowable rate.

In another feature and advantage of embodiments, when a within-time continuous infusion is programmed, wherein delivery is interrupted and the recalculated rate is higher than the maximum rate, the pump or one or more associated modules is configured to use the maximum delivery rate when delivery is restarted and further to allow the delivery time to extend beyond the set time.

In another feature and advantage of embodiments, when a flush is programmed for a within-time continuous infusion, the pump or one or more associated modules is configured to include the flush volume with the dosage when calculating the delivery rate.

In another feature and advantage of embodiments, when a flush is programmed for a within-time continuous infusion, the pump or one or more associated modules is configured to reduce the time used for calculating the delivery rate by a preset time associated with changing the syringe. In other embodiments, the total time used for calculating the delivery rate can be reduced by a varying amount of time, depending on the type of syringe. In other embodiments, the total time used for calculating the delivery rate can be reduced by a time programmed by the user.

Regardless of delivery mode, whether continuous, intermittent, or defined by the user, embodiments herein are not limited to the example modes provided. The above summary is not necessarily intended to describe each illustrated embodiment or every implementation of the subject matter hereof. The figures and the detailed description that follow more particularly exemplify these embodiments.

### BRIEF DESCRIPTION OF THE DRAWINGS

Subject matter hereof may be more completely understood in consideration of the following detailed description of various embodiments of the subject matter in connection with the accompanying drawings, in which:

FIG. 1A is a perspective view of a syringe type infusion pump, according to an embodiment.

FIG. 1B is a front view of a control module of an ambulatory type infusion pump, according to an embodiment.

FIG. 2 is a block diagram of an infusion pump system, according to an embodiment.

FIG. 3 is a block diagram of a pump control subsystem, according to an embodiment.

FIG. 4 is an annotated graph showing flow rate against time in an infusion controlled by the pump control subsystem of FIG. 3, according to an embodiment.

FIG. 5 is an annotated graph showing flow rate against time in an infusion controlled by the pump control subsystem of FIG. 3, according to an embodiment.

FIG. 6 is an annotated graph showing flow rate against time in an infusion controlled by the pump control subsystem of FIG. 3, according to an embodiment.

FIG. 7 is a flowchart of a method for controlling infusions in an infusion pump with a within-time infusion mode, according to an embodiment.

FIG. 8 is a flowchart of a method for managing volume infused in an infusion pump with a within-time infusion mode, according to an embodiment.

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION OF THE DRAWINGS

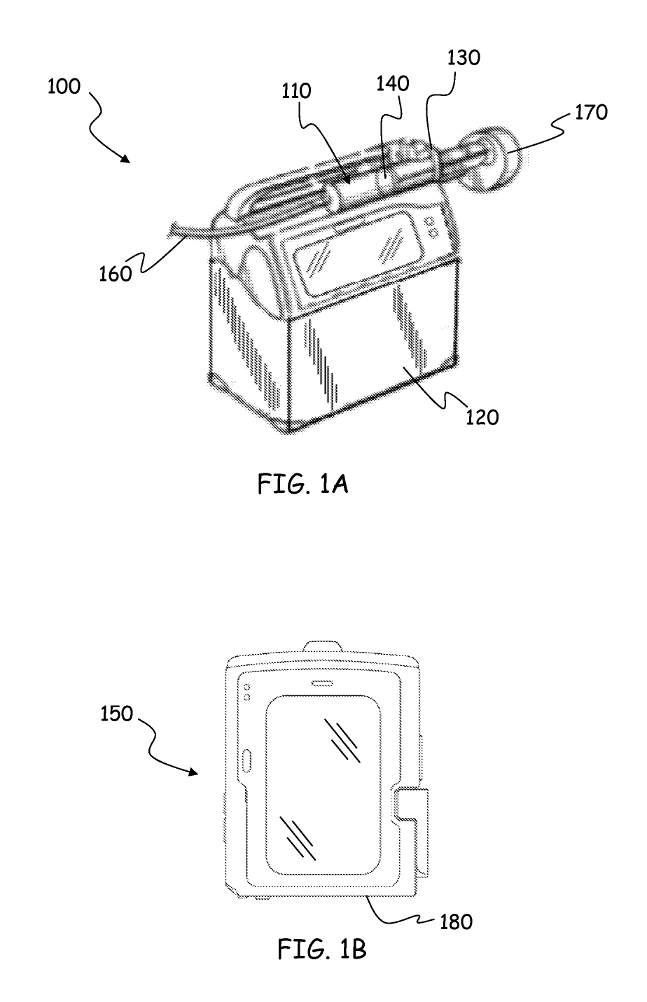

FIGS. 1A and 1B show examples of infusion pumps 100 and 150, respectively (also referred to more generally in this disclosure by numeral 100), which can be used to implement embodiments of the systems and methods discussed herein. In general, infusion pump 100 is a syringe-type pump that can be used to deliver a wide range of infusates, drug therapies and treatments. Infusion pump 100 includes a pharmaceutical container or syringe 110, which is supported on and secured to housing 120 by clamp 130, respectively. In embodiments, syringe 110 can be separately supplied from pump 100. In other embodiments, syringe 110 is an integrated component of pump 100. Syringe 110 includes a plunger 140 that forces fluid outwardly from syringe 110 via infusion line 160 that is connected to a patient. A motor and lead screw arrangement internal to housing 120 of pump 100 cooperatively actuates a pusher or plunger driver mechanism 170, to move plunger 140 of syringe 110. In embodiments, a sensor (not shown; which is typically internal to plunger driver mechanism 170) monitors force and/or plunger position in the syringe according to system specifications.

Infusion pump 150 shown in FIG. 1B is an example of an ambulatory-type infusion pump that can be used to deliver a wide range of infusates, drug therapies, and treatments. Such ambulatory pumps can be comfortably worn by or otherwise removably coupled to a user for in-home ambulatory care by way of belts, straps, clips or other simple fastening means, and can also be alternatively provided in ambulatory pole-mounted arrangements within hospitals and other medical care facilities. Infusion pump 150 generally includes a peristaltic type infusion pump mechanism that controls the flow of medication from a reservoir (not shown in FIG. 1B) of fluid coupled to pump 150 through a conduit from the reservoir which can matingly pass along bottom surface 180 of pump 150. The reservoir can comprise a cassette that is attached to the bottom of pump 150 at surface 180, or an IV bag or other fluid source that is similarly connected to pump 150 via an adapter plate (not shown) at surface 180. Specifically, pump 150 uses valves and an expulsor located on bottom surface 180 to selectively squeeze a tube of fluid (not shown) connected to the reservoir to effect the movement of the fluid supplied by the reservoir through the tube and to a patient in peristaltic pumping fashion. Infusion pumps 100 and 150 are two examples of infusion pumps that can be suitable for use with embodiments discussed herein, though other pumps and devices can be used in other embodiments of infusion systems utilizing subject matter hereof.

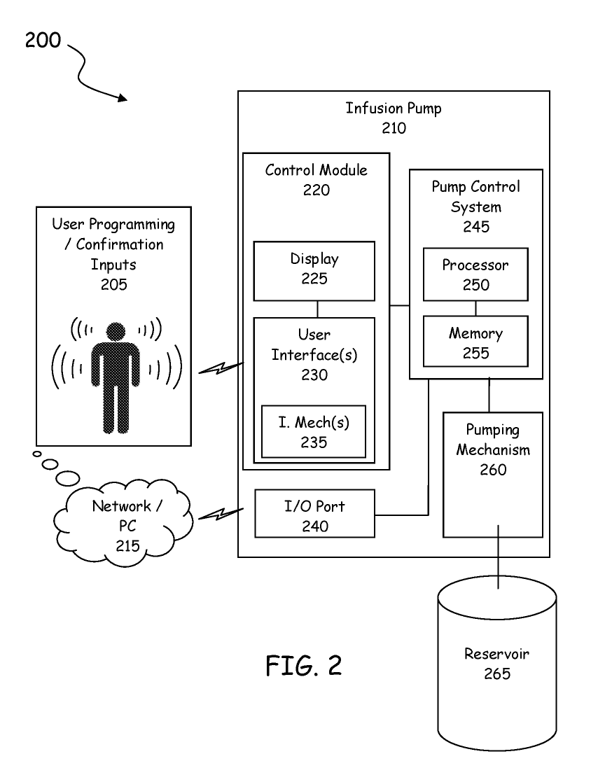

FIG. 2 is a schematic diagram of an infusion pump system 200. System 200 includes infusion pump 210 having pump control system 245 with processor 250 and memory 255 programmable with selected protocols, profiles, segments of profiles, and other settings for controlling operation of pumping mechanism 260 such as, for example, the aforementioned syringe and ambulatory or peristaltic type mechanisms.

In an embodiment, processor 250 can be any programmable device that accepts digital data as input, is configured to process the input according to instructions or algorithms, and provides results as outputs. In an embodiment, processor 250 can be a central processing unit (CPU) configured to carry out the instructions of a computer program. In another embodiment, processor 250 can be an application specific integrated circuit (ASIC). In another embodiment, processor 250 can be a field-programmable gate array (FPGA). Processor 250 is therefore configured to perform arithmetical, logical, and input/output operations.

Memory 255 can comprise volatile or non-volatile memory as required by the coupled processor 255 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of subject matter hereof.

Infusion pump 200 can also include control module 220 (e.g., a user interface) for relaying commands to pump control system 245. Control module 220 includes at least one user interface 230 utilizing operator input technology including input mechanism(s) 235, which work with display 225. In some cases display 225 will be considered part of user interface(s) 230. User interface 230 generally allows a user to enter various parameters, including but not limited to names, drug information, limits, delivery shapes, information relating to hospital facilities, as well as various user-specific parameters (e.g., patient age and/or weight). Infusion pump 210 can include USB port or other appropriate input/output (I/O) interface port 240 for connecting infusion pump 210 to network or computer 215 having software designed to interface with infusion pump 210. Power to infusion pump 210 is accomplished via an AC or DC power cord or an internally provided battery source. Embodiments can also include a wireless power source.

User inputs 205 to the system can be provided by programming from a user, such as a patient, pharmacist, scientist, drug program designer, medical engineer, nurse, physician, or other medical practitioner or healthcare provider. User inputs 205 may utilize direct interfacing (i.e., a keyboard or other touch-based inputs) or user inputs 205 may utilize indirect or “touchless” interfacing (i.e., gestures; voice commands; facial movements or expressions; finger, hand, head, body and arm movements; or other inputs that do not require physical contact). User inputs 205 are generally interfaced, communicated, sensed, and/or received by operator input mechanisms 235 of user interface 230. Operator input mechanisms 235 may include, for example, keyboards, touch screens, cameras, or sensors of electric field, capacitance, or sound. As depicted in FIG. 2, infusion pump 210 is operably coupled to reservoir 265 via pumping mechanism 260. In embodiments, reservoir 265 can comprise any suitable infusate supply, such as an IV bag, syringe, continuous supply, or other infusate storage. In an embodiment, reservoir 265 is coupled to pumping mechanism 260 by cannula suitable for transferring infusate stored in reservoir 265 to pumping mechanism 260.

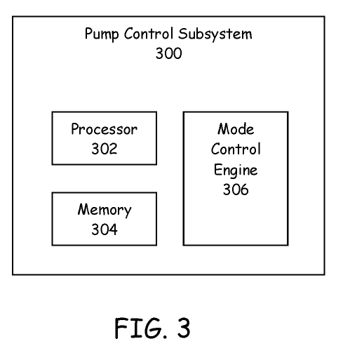

Referring to FIG. 3, a pump control subsystem 300 is configured for controlling operation of the pumping mechanisms of an infusion pump; for example, infusion pumps 100, 150, and 210. In an embodiment, pump control subsystem 300 generally comprises processor 302, memory 304, and mode control engine 306. In an embodiment, pump control subsystem 300 can be substantially similar to pump control system 245 as depicted in FIG. 2. For example, processor 302 and memory 304 can be substantially similar to processor 250 and memory 255 as described with respect to pump control system 245.

Mode control engine 306 is configured for the programming and scheduling of infusions, as described herein. In an embodiment, mode control engine 306 is configured to automatically and dynamically adjust an infusion rate to meet scheduled delivery time and rate limits. In embodiments, a flush can also be included or considered in the scheduled delivery time. In embodiments, mode control engine can include a timing module. The timing module can comprise a counter, scheduler, calendar, or other suitable timing components that can be used in calculating infusion parameters related to rate and time. In an embodiment, mode control engine 306 is operably coupled to processor 302 and/or memory 304.

Mode control engine 306 can be constructed, programmed, configured, or otherwise adapted, to autonomously carry out a function or set of functions. The term engine as used herein is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that cause the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly in parallel or series with, and/or complementary to other functions, or distributed differently among a set of engines than specifically illustrated in the examples herein.

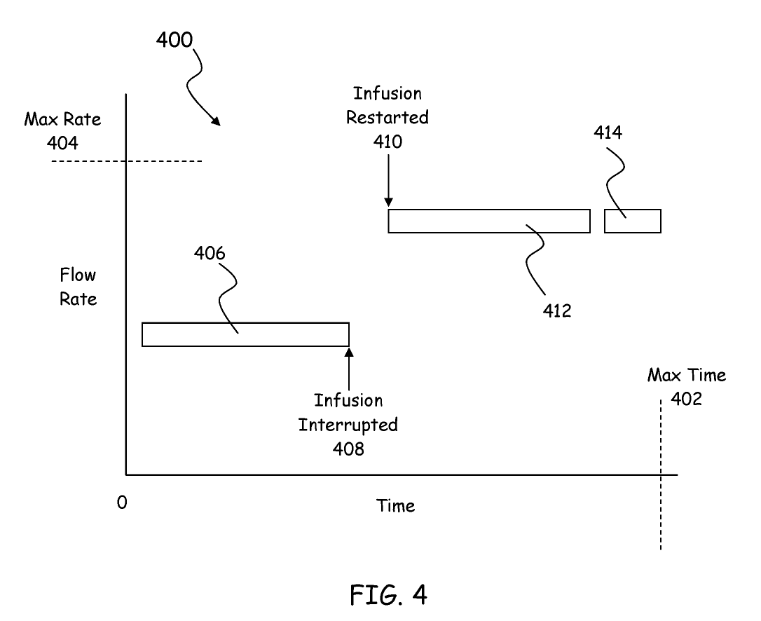

Referring to FIG. 4, an annotated graph 400 showing flow rate against time in an infusion controlled by a pump control system or subsystem (collectively, “subsystem”) is depicted. In an embodiment, the pump control subsystem is pump control subsystem 300, whereby mode control engine 306 is configured for a within-time infusion mode. In another embodiment, the pump control subsystem is pump control system 245. Graph 400 depicts an infusion process being interrupted and restarted. In the embodiment depicted, as will be described, the infusion rate is automatically and dynamically varied upon restarting to meet a scheduled delivery time.

In FIG. 4, at time 0, the pump control subsystem is programmed for a within-time infusion. For example, referring to FIG. 2 and infusion pump 210, control module 220 can be utilized by a physician, nurse, other medical professional, or by the patient to relay commands to pump control system 245. In an embodiment, user interface 230 can accept operator input with one or more input mechanisms 235, in combination with display 225. For ease of explanation in this example embodiment, and with reference now to FIG. 3, pump control subsystem 300 and mode control engine 306 will be referred to as the pump control subsystem, but one skilled in the art will readily appreciate that the within-time infusion modes described herein can be applied to any applicable pump and pump control subsystem and programming by any suitable control module.

Utilizing a control module, for example, and referring again to FIG. 2, control module 220, an operator can specify a target delivery time and a rate limit, along with the desired infusion. While a single infusion is described herein, multiple infusions can likewise be specified. For example, referring to FIG. 4, max time 402 can reflect the target delivery time. Max rate 404 can reflect the rate limit. In embodiments, max time 402 and max rate 404 can be specified by user input, predefined or preprogrammed, or otherwise derived from one or more databases or drug libraries.

An original infusion 406 begins some time after the pump is programmed. Original infusion 406 is executed by the pump for a duration of time. At 408, original infusion 406 is interrupted during that duration. As shown, the infusion is restarted at 410 as a restarted infusion 412.

During the time between when original infusion 406 is interrupted at 408 and the infusion restarted at 410 as restarted infusion 412, pump control subsystem 300 is configured to recalculate the delivery rate in order to complete the delivery within max time 402, without exceeding the maximum allowable rate 404. As shown, restarted infusion 412 is at a higher rate than original infusion 406 due to the shortened time in which the infusion can be delivered, as limited by max time 402. In an embodiment, the delivery rate recalculation can be made for any point in time along the time axis after the infusion is interrupted at 408. In other embodiments, the delivery rate recalculation can be made instantaneously at the time the infusion is commanded (or otherwise programmed) to be restarted. As such, the delivery rate is automatically and dynamically varied upon restart. In other embodiments, options for restart delivery rate can be calculated and presented to the operator.

A flush 414 can optionally be added to the end of the infusion. In the example of FIG. 4, for the interrupted infusion at 408, flush 414 is accounted for at the end of restarted infusion 412 to be added to the total time duration for the infusion. For example, pump control subsystem 300 can be configured to include the flush volume with the dosage when calculating the delivery rate. In an example a delay exists between restarted infusion 412 and flush 414 to account for the time the reservoir is changed out to contain a flush fluid. In embodiments, the time delay for the reservoir change is preplanned so as to not cause a rate spike. In embodiments, pump control subsystem 300 is further configured to reduce the time used for calculating the delivery rate by a preset time associated with changing the syringe.

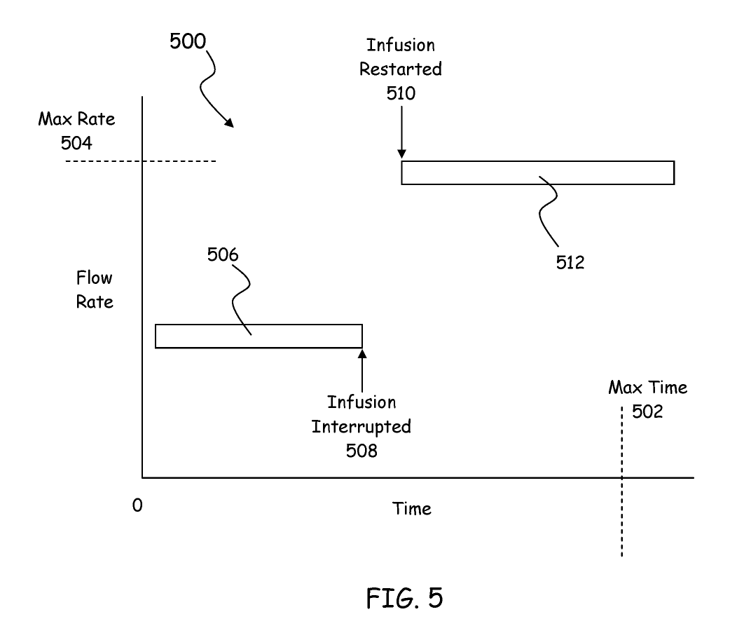

Referring to FIG. 5, an annotated graph 500 showing flow rate against time in an infusion controlled by a pump control subsystem is depicted. Graph 500 depicts a single infusion being interrupted and restarted. In the embodiment depicted, as will be described, the infusion rate is automatically and dynamically varied upon restarting to the maximum rate allowed in view of a scheduled delivery time.

Utilizing the control module, an operator can specify a target delivery time and a rate limit, along with the desired infusion. For example, referring to FIG. 5, max time 502 can reflect the target delivery time. As will be described, in an embodiment, max time 502 is only a soft limit, as it can be exceeded. Max rate 504 can reflect the rate limit. In embodiments, max time 502 and max rate 504 can be specified by user input, predefined or preprogrammed, or otherwise derived from one or more databases or drug libraries.

An original infusion 506 begins some time after the pump is programmed. Original infusion 506 is executed by the pump for a duration of time. At 508, original infusion 506 is interrupted during that duration. As shown, the infusion is restarted at 510 as a restarted infusion 512. In this embodiment, no flush is added to the end of the infusion.

During the time between when original infusion 506 is interrupted at 508 and the infusion restarted at 510 as restarted infusion 512, pump control subsystem 300 is configured to recalculate the delivery rate in view of the delivery within max time 502, without exceeding the maximum allowable rate 504. As shown, restarted infusion 512 is set at the maximum rate allowed for the infusion. In an embodiment, as shown in FIG. 5, pump control subsystem 300 is configured to calculate that the restart rate, if implemented due to the shortened time period for delivery caused by interruption 508, would be higher than maximum rate 504. As a result, pump control subsystem 300 is configured to set restarted infusion 512 at maximum delivery rate 504 when delivery is restarted and allow the delivery time to extend beyond max time 502.

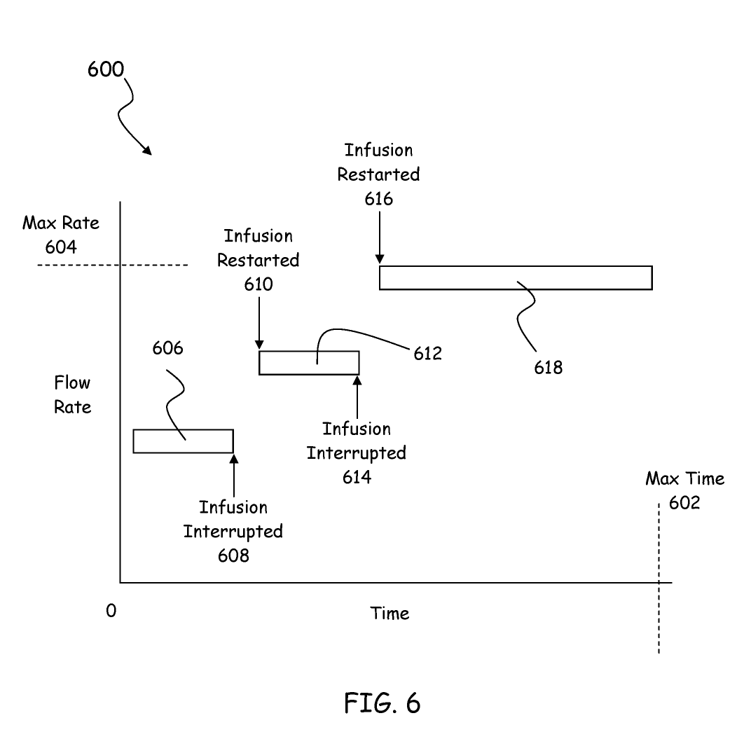

Referring to FIG. 6, an annotated graph 600 showing flow rate against time in an infusion controlled by a pump control subsystem is depicted. Graph 600 depicts a single infusion procedure being interrupted twice and restarted twice. In the embodiment depicted, as will be described, the infusion rate is automatically and dynamically varied upon restarting to the maximum rate allowed to meet a scheduled delivery time.

Utilizing the control module, an operator can specify a target delivery time and a rate limit, along with the desired infusion. For example, referring to FIG. 6, max time 602 can reflect the target delivery time. Max rate 604 can reflect the rate limit. In embodiments, max time 602 and max rate 604 can be specified by user input, predefined or preprogrammed, or otherwise derived from one or more databases or drug libraries.

An original infusion 606 begins some time after the pump is programmed. Original infusion 606 is executed by the pump for a duration of time. At 608, original infusion 606 is interrupted during that duration. As shown, the infusion is restarted at 610 as a first restarted infusion 612.

During the time between when original infusion 606 is interrupted at 608 and the infusion restarted at 610 as first restarted infusion 612, pump control subsystem 300 is configured to recalculate the delivery rate in view of the delivery within max time 602, without exceeding the maximum allowable rate 604. As shown, first restarted infusion 612 is at a higher rate than original infusion 606 due to the shortened time in which the infusion can be delivered, as limited by max time 602.

First restarted infusion 612 begins as automatically and dynamically scheduled, according to the pump control subsystem. First restarted infusion 612 is executed by the pump for a duration of time. At 614, first restarted infusion 612 is interrupted during that duration. As shown, the infusion is restarted at 616 as a second restarted infusion 618.

During the time between when first restarted infusion 612 is interrupted at 614 and the infusion restarted at 616 as second restarted infusion 618, pump control subsystem 300 is configured to recalculate the delivery rate in view of the delivery within max time 602, without exceeding the maximum allowable rate 604. As shown, second restarted infusion 618 is set at the maximum rate allowed for the infusion.

The timelines provided in FIGS. 4-6 are for illustration only and are not intended to be limiting or to define the scope of pump control subsystems in executing within-time infusion modes. Rather, one skilled in the art will readily appreciate that any number of pump control subsystems are possible within the scope of the novel and inventive subject matter hereof.

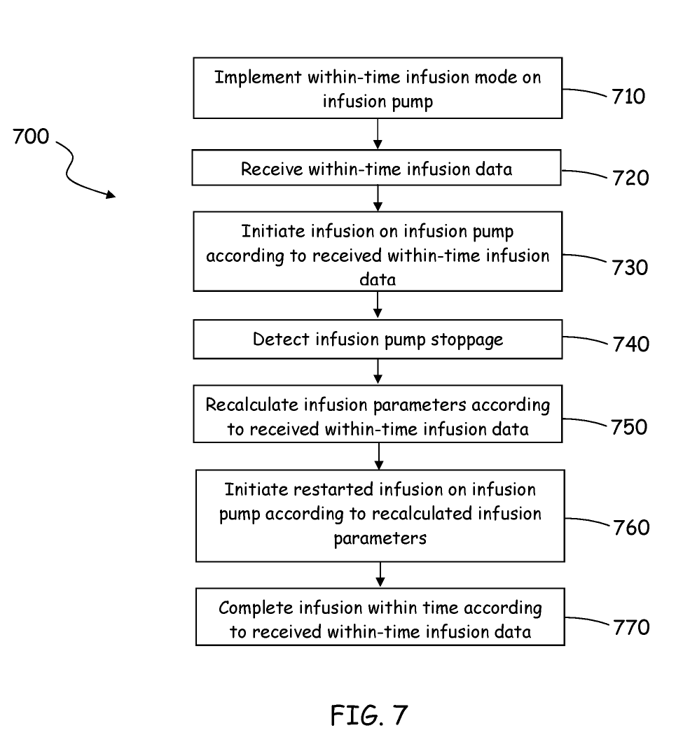

Referring to FIG. 7, a flowchart of a method 700 for controlling infusions in an infusion pump according to a within-time infusion mode is depicted. Method 700 can be implemented by, for example, pump control system 245 and/or subsystem 300.

At 710, a within-time infusion mode is implemented on an infusion pump. For example, mode control engine 306 can be instantiated in hardware and/or software as a set of program instructions that adapt mode control engine 306 to implement the within-time infusion mode functionality. Practically, mode control engine 306 software can be installed on the infusion pump. In other embodiments, a pump can be powered on or restarted so that mode control engine 306 is active. In still other embodiments, mode control engine 306 comprising hardware and software can be retrofitted onto an existing pump or be in communication with the pump remotely such as via a wired or wireless connection with, for example, a server system.

At 720, within-time infusion data is received by mode control engine 306. For example, as described above, within-time infusion data can be input into the pump through a user interface, which can accept operator input with one or more input mechanisms in combination with a user interface such as a display screen. In an embodiment, the received within-time infusion data can comprise a target delivery time, a rate limit, and the desired infusion. In other embodiments, a delivery time can comprise a “hard limit” maximum time for the infusion. In embodiments, other data suitable for deriving a within-time infusion can be received.

In an example of a within-time intermittent infusion, a maximum time within which the delivery should be completed can be received by mode control engine 306. Further, in an example, when a within-time intermittent infusion is programmed, a maximum allowable delivery rate can be received by mode control engine 306.

In another example, for a within-time continuous infusion, a planned time within which the delivery should be completed can be received by mode control engine 306. Further, in an example, when a within-time continuous infusion is programmed, a maximum allowable delivery rate can be received by mode control engine 306.

At 730, an infusion is initiated on the infusion pump in accordance with the received within-time infusion data. For example, an infusion can be commanded that comprises a continuous or intermittent infusion to be completed by the received target delivery time, and to be administered below the received infusion rate limit. In embodiments, multiple infusions can be commanded according to the received within-time infusion data. Pump control system 300 and any appropriate combination of processor 302, memory 304, and mode control engine 306 can be programmed for the particular infusion or multiple infusions commanded by the received within-time infusion data.

In an example, when a within-time intermittent infusion is initiated or programmed, mode control engine 306 is configured to calculate the delivery rate based on the dose to be delivered and the amount of time during which the delivery should occur.

In another example, when a flush is programmed for a within-time intermittent infusion, mode control engine 306 is configured to include the flush volume with the dosage when calculating the delivery rate.

In another example, when a flush is programmed for a within-time intermittent infusion, mode control engine 306 can reduce the time used for calculating the delivery rate by a preset time for changing the syringe during the flush procedure. In another example, when a clinician pushes the flush manually, mode control engine 306 can allow the clinician to enter the flush amount to add to the delivery total.

In another example, when a within-time continuous infusion is programmed, mode control engine 306 can calculate the delivery dose to be delivered based on the rate and the amount of time during which the delivery should occur.

In another example, when a flush is programmed for a within-time continuous infusion, mode control engine 306 can include the flush volume with the dosage when calculating the delivery rate.

In another example, when a flush is programmed for a within-time continuous infusion, mode control engine 306 can reduce the time used for calculating the delivery rate by a preset time for changing the syringe.

At 740, a stoppage of the infusion pump is detected. For example, pump control subsystem 300 can receive a signal from the operably coupled pumping mechanism that a stoppage has occurred. In an example, the pumping mechanism can indicate that an occlusion has occurred. In embodiments, pump control subsystem 300 can receive a signal from other pump components of an error, stoppage, or other abnormal or unintended condition. In other embodiments, pump control subsystem 300 can actively monitor the pumping mechanism and/or other pump components.

At 750, the infusion parameters are recalculated according to the received within-time infusion data in view of the new timeline for completion after the stoppage. In an embodiment, mode control engine 306 comprises multiple algorithms for handling the variations in stoppage and timing. In an embodiment, a timing module is configured to determine a difference between the start time and the received target delivery time. For example, in a pump stoppage after 20 minutes in a within-time infusion target of 30 minutes, the timing module can calculate 10 minutes remaining to complete the infusion. In embodiments, the timing module can further incorporate the delayed or stopped time into the calculation. For example, in a pump stoppage after 20 minutes and a stopped delay of 5 minutes in a within-time infusion target of 30 minutes, the timing module can calculate 5 minutes remaining to complete the infusion. In embodiments, infusion parameters are recalculated when the pump is initiated for a restarted infusion at 760. The timing module and/or mode control engine 306 can utilize pump counters, CPU clocks, date-time stamps, or any other suitable mechanism for time determination.

In another example, when a within-time intermittent infusion is programmed and delivery is interrupted, mode control engine 306 can recalculate the delivery rate when delivery is restarted in order to complete the delivery within the time specified without exceeding the maximum allowable rate.

In another example, when a within-time intermittent infusion is programmed and delivery is interrupted and the recalculated rate is higher than the maximum rate, mode control engine 306 can use the maximum delivery rate when delivery is restarted and allow the delivery time to extend beyond the set time or target time.

In another example, when a within-time continuous infusion is programmed and delivery is interrupted, mode control engine 306 can recalculate the delivery rate when delivery is restarted in order to complete the calculated dose to be delivered within the time specified and without exceeding the maximum allowable rate.

In another example, when a within-time continuous infusion is programmed, delivery is interrupted, and the recalculated rate is higher than the maximum rate, mode control engine 306 can use the maximum delivery rate when delivery is restarted and allow the delivery time to extend beyond the set time or target time.

At 760, the infusion is restarted on the infusion pump in accordance with the recalculated infusion parameters. In embodiments, as described above, the calculations at 750 are performed when the user restarts the pump. In such embodiments, the pump is therefore considered to be restarted at time 0 with respect to the time remaining in order to calculate a new rate. In embodiments, the rate for the restarted infusion is greater than the rate for the original infusion, as described herein. In other embodiments, if the remaining time for infusion is determined to be sufficient, the pump control system 300 can restart the infusion at the same rate as the original infusion.

At 770, the infusion is completed within the specified time, according to the received within-time infusion data. In an embodiment, the infusion is completed within a maximum (hard limit) time. In another embodiment, the infusion is completed within a maximum (soft limit) time. In other embodiments, the infusion is completed outside of the soft limit time.

According to embodiments, smart piggybacking of infusions can be implemented. In traditional piggybacking, as known to medical practitioners, two separate reservoirs share a common infusion line through one pump input. After the rate is set by the pump, the respective height of the reservoirs according to simple fluid height mechanics determines which bag empties first. For example, a first reservoir will flow first when placed relatively higher than a second reservoir.

In an embodiment, multiple drug or multiple reservoir “piggybacking” comprises a mechanical implementation for delivering two drugs or fluids at a programmed total volume and specific rate. In certain situations, one or both of the multiple reservoirs need to be delivered within a particular time. In embodiments, large volume pumps (LVP) can be utilized in smart piggybacking.

In an embodiment, an infusion pump can implement a within-time delivery mode configured for a primary infusion and a secondary infusion, sometimes known in the art as “piggybacking.” In an embodiment, the within-time delivery mode can determine which of the reservoirs is being depleted and adjust the infusion parameters for the other reservoir accordingly. Depending on the rate of the infusion, the rate of infusion of the second reservoir can be adjusted according to the within-time algorithms described herein. Embodiments can therefore determine when the first reservoir is completed. Embodiments can also automatically adjust the second reservoir infusion rate. For example, the infusion parameters can be adjusted according to the first reservoir completion time and the overall completion time.

In an “unplanned” secondary infusion or unplanned “piggybacking,” a continuous infusion can be administered to a patient from a first reservoir through an infusion pump. A patient may be receiving, for example, an hour-long continuous first infusion from the first reservoir. During the time of the continuous infusion, the patient may need a second infusion. In certain situations, it may not be possible to administer the second infusion, due to shortages in pump hardware or infusion lines to the patient. In such situations, a second reservoir can be operably coupled to a common infusion line with the first reservoir. In embodiments, an infusion pump implementing a within-time delivery mode including a primary infusion and a secondary infusion can automatically adjust the infusion rate of the combined infusion, or the remaining primary infusion, once the unplanned piggybacking has completed. In other embodiments, the within-time delivery mode can recommend rate changes to be accepted by an administering medical professional.

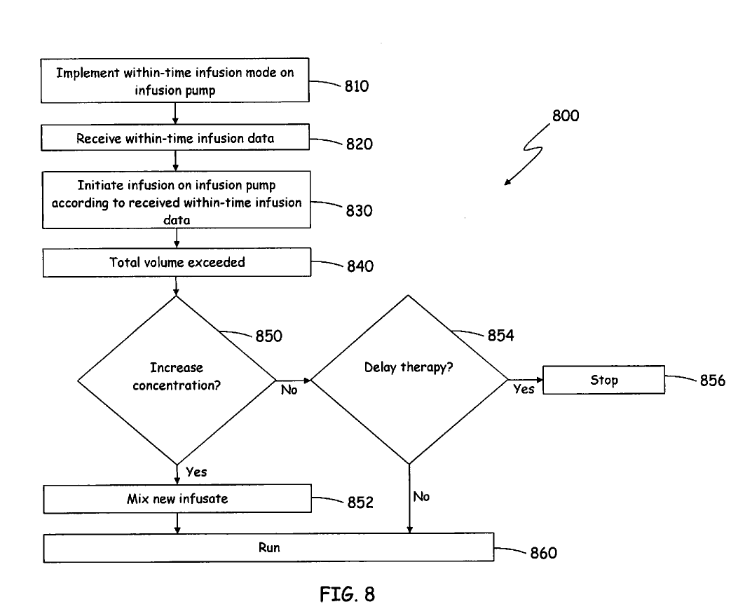

In another embodiment, referring to FIG. 8, a flowchart of a method 800 for managing volume infused in an infusion pump with a within-time infusion mode is depicted.

At 810, a within-time infusion mode is implemented on an infusion pump. For example, mode control engine 306 can be instantiated in hardware and/or software as a set of program instructions that adapt mode control engine 306 to implement the within-time infusion mode functionality. Practically, mode control engine 306 software can be installed on the infusion pump. In other embodiments, a pump can be powered on or restarted so that mode control engine 306 is active. In still other embodiments, mode control engine 306 comprising hardware and software can be retrofitted onto an existing pump or be in communication with the pump remotely such as via a wired or wireless connection with, for example, a server system.

At 820, within-time infusion data is received by mode control engine 306. For example, as described above, within-time infusion data can be input into the pump through a user interface, which can accept operator input with one or more input mechanisms in combination with a user interface such as a display screen. In an embodiment, the received within-time infusion data can comprise a total volume limit and a target delivery time or a delivery time limited by a hard limit.

At 830, an infusion is initiated on the infusion pump in accordance with the received within-time infusion data. In an embodiment, the infusion is initiated particularly with the total volume limit and target delivery time or delivery time limited by a hard limit.

At 840, in an embodiment, the within-time infusion mode calculates that the total volume limit will be exceeded. In an embodiment, this determination can be made after delivery is started. In another embodiment, the infusion is not initiated at 830, but instead, the method 800 proceeds to 840 to evaluate the received within-time infusion data so that any received total volume limit, target delivery time, or delivery time limited by a hard limit are met prior to delivery being started.

At 850, a decision point is reached related to the concentration level of the infusion drug. In an embodiment at 850, the user can be prompted that the total volume limit is exceeded or will be exceeded. The user can likewise be prompted at 850 to confirm an increase of the concentration level of the infusate. In another embodiment, the infusion concentration can be automatically increased.

From 850, method 800 can proceed by increasing the medication concentration (“Yes”). Increasing the medication concentration can be achieved by any suitable means; for example, by substituting a new reservoir for the existing reservoir. One skilled in the art will readily understand that the medication concentration can be adjusted by any number of methods or procedures. Accordingly, at 852, a new mix of infusate is created with the increased medication concentration. In an embodiment, the new mix at 852 can be made according to input received at 850. In another embodiment, the new mix at 852 can be made automatically according to the previously-received total volume limit, target delivery time, or delivery time.

At 860, the infusion is run with the increased concentration to meet the target delivery time or delivery time limited by a hard limit.

Alternatively from 850, method 800 can proceed to 854 without increasing the medication concentration (“No”). At 854, a decision point whether to delay the therapy is reached. In an embodiment at 854, the user can be prompted to decide whether the therapy should be delayed. In other embodiments, a delay decision can be pre-programmed into method 800 such that method 800 checks the pre-programmed value to determine the path from 854. From 854, if the therapy is to be delayed (“Yes”), method 800 proceeds to stop any further infusion at 856.

Alternatively from 854, if there is no delay desired (“No”), the infusion is run at 860 without an increased concentration and without a delay to extend beyond the target delivery time or delivery time limited by a hard limit.

Various embodiments of systems, devices, and methods have been described herein. These embodiments are given only by way of example and are not intended to limit the scope of subject matter hereof. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized commensurate with the scope of subject matter hereof.

Persons of ordinary skill in the relevant arts will recognize that subject matter hereof may comprise fewer features than illustrated in any individual embodiment described above. The embodiments described herein are not meant to be an exhaustive presentation of the ways in which the various features of subject matter hereof may be combined. Accordingly, the embodiments are not mutually exclusive combinations of features; rather, the subject matter hereof may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims of subject matter hereof, it is expressly intended that the provisions of Section 112, sixth paragraph of 35 U.S.C. are not to be invoked unless the specific terms “means for” or “step for” are recited in a claim.

1. An infusion pump comprising:
a control module configured to receive within-time infusion mode pump parameters, wherein one of the within-time infusion mode pump parameters is a completion time for an infusion;
a pump control subsystem comprising a mode control engine configured to implement the within-time infusion mode pump parameters, including calculating an infusion restart rate based on at least the completion time for the infusion if the infusion pump becomes stopped; and
a pumping mechanism operably coupled to a reservoir and configured to be controlled by the pump control subsystem.

2. The infusion pump of claim 1, wherein the control module comprises:
a pump display configured for displaying data related to the pump control subsystem; and
an input mechanism configured for inputting the within-time infusion mode pump parameters.

3. The infusion pump of claim 1, wherein the completion time is one of a hard limit maximum completion time or a soft limit desired time.

4. The infusion pump of claim 1, wherein one of the within-time infusion mode pump parameters is a maximum infusion rate.

5. The infusion pump of claim 4, wherein the infusion restart rate is calculated to be at a level below the maximum infusion rate.

6. The infusion pump of claim 4, wherein if the infusion restart rate is calculated to be higher than the maximum infusion rate, the infusion restart rate is set to the maximum infusion rate and the infusion is delivered beyond the completion time.

7. The infusion pump of claim 1, wherein one of the within-time infusion mode pump parameters is a flush volume and the mode control engine is configured to calculate an infusion restart rate based at least on the flush volume.

8. The infusion pump of claim 7, wherein the mode control engine is configured to reduce the completion time used for calculating the rate by a syringe changing time.

9. The infusion pump of claim 7, wherein the flush volume is input manually to the control module.

10. The infusion pump of claim 1, wherein the pump control subsystem comprises:
a processor configured to carry out the instructions of the mode control engine; and
memory operably coupled to the processor,
wherein the mode control engine is operably coupled to the processor and the memory.

11. A method for controlling an infusion in an infusion pump, the method comprising:
implementing a within-time infusion mode on an infusion pump via a mode control engine;
receiving within-time infusion mode pump data, wherein the within-time infusion mode pump data includes a completion time;
initiating an infusion on the infusion pump according to infusion parameters derived from the received within-time infusion mode pump data;
detecting a stoppage of the infusion pump;
calculating an infusion restart rate based on at least the completion time for the infusion;
restarting the infusion on the infusion pump according to the calculated infusion restart rate; and
completing the restarted infusion within the completion time.

12. The method for controlling an infusion in an infusion pump of claim 11, wherein the completion time is one of a hard limit maximum completion time or a soft limit desired time.

13. The method for controlling an infusion in an infusion pump of claim 11, wherein the within-time infusion mode pump data includes a maximum infusion rate.

14. The method for controlling an infusion in an infusion pump of claim 13, wherein the infusion restart rate is calculated to be at a level below the maximum infusion rate.

15. The method for controlling an infusion in an infusion pump of claim 13, wherein if the infusion restart rate is calculated to be higher than the maximum infusion rate, the infusion restart rate is set to the maximum infusion rate and the infusion is delivered beyond the completion time.

16. The method for controlling an infusion in an infusion pump of claim 11, wherein the within-time infusion mode pump data includes a flush volume and calculating an infusion restart rate is based at least on the flush volume.

17. The method for controlling an infusion in an infusion pump of claim 16, further comprising reducing the completion time used for calculating the rate by a syringe changing time.

18. The method for controlling an infusion in an infusion pump of claim 16, wherein the flush volume is input manually to the infusion pump.

19. The method for controlling an infusion in an infusion pump of claim 11, wherein detecting the stoppage of the infusion pump comprises detecting an occlusion.

20. A pump control system comprising:
programmable circuitry configured to:
implement a within-time infusion mode on an infusion pump via a mode control engine;
receive within-time infusion mode pump data, wherein the within-time infusion mode pump data includes a completion time;
initiate an infusion on the infusion pump according to infusion parameters derived from the received within-time infusion mode pump data;
detect a stoppage of the infusion pump;
calculate an infusion restart rate based on at least the completion time for the infusion;
restart the infusion on the infusion pump according to the calculated infusion restart rate; and
complete the restarted infusion within the completion time.
